(19) United States
(12) Patent Application Publication
(10) Pub. No.: US 2026/0085345 A1
(43) Pub. Date: Mar. 26, 2026

(54) MICROSPHERE FORMULATION FOR NUCLEIC ACID AMPLIFICATION, AMPLIFICATION METHOD, AND USE IN JOINT DETECTION

(71) Applicant: SHANGHAI BIOGERM MEDICAL TECHNOLOGY CO., LTD., Shanghai (CN)

(72) Inventors: Pei LI, Shanghai (CN); Baihui ZHAO, Shanghai (CN); Chunyan LI, Shanghai (CN)

(21) Appl. No.: 19/111,475

(22) PCT Filed: Dec. 30, 2022
(86) PCT No.: PCT/CN2022/144231

§ 371 Date: Mar. 13, 2025

(30) Foreign Application Priority Data
Sep. 16, 2022 (CN) 202211133161.9

(51) Int. Cl. C12Q 1/6848; C12Q 1/70

(52) U.S. Cl. CPC C12Q 1/6848; C12Q 1/701

(57) ABSTRACT

The present disclosure relates to the technical field of molecular diagnosis, and particularly, to a microsphere formulation for nucleic acid amplification, an amplification method, and use in joint detection. The disclosed microsphere formulation can be stored for a long duration at 2-8° C. When the microsphere preparation is used, no solvent is additionally added, and the microsphere formulation is directly mixed with a sample to be detected. On the premise that the concentration of an original system is not changed, the template content in the system can be remarkably improved, so that the detection sensitivity is improved. By using the microsphere formulation in recombinase polymerase amplification (RPA), recombinase-aided amplification (RAA), or a double or multiple detection formed by combining a second reaction on the basis of RPA or RAA, the sensitivity can be remarkably improved, and meanwhile, the amplification specificity is ensured.

### CROSS-REFERENCE TO RELATED APPLICATION(S)

The present disclosure claims priority to the Chinese Patent Application No. CN202211133161.9 entitled “MICROSPHERE FORMULATION FOR NUCLEIC ACID AMPLIFICATION, AMPLIFICATION METHOD, AND USE IN JOINT DETECTION” and filed with China National Intellectual Property Administration on Sep. 16, 2022, which is incorporated herein by reference in its entirety.

### TECHNICAL FIELD

The present disclosure relates to the technical field of molecular diagnosis, and particularly to a microsphere preparation for nucleic acid amplification, an amplification method, and use in combined assays.

### BACKGROUND

With the development of diagnostic technologies, molecular diagnostic technology has gradually become an important detection means for diagnosing diseases in human daily life, and with the diversification of detection targets, the application of multiplex detection is more and more extensive. In order to ensure sufficient accuracy, most multiplex detection methods require amplification of the target fragments, and the amplified products are then used for detection.

Presently, the amplification reaction system of the target to be detected can function well only in an aqueous phase environment, and the reagents traditionally used for amplification are basically stored and transported in an aqueous form. A limited amount of existing types of microsphere preparation for amplification needs to be redissolved using solvents into a solution form prior to carrying out an amplification reaction, and adopting the microsphere preparation form aims only to improve the stability and reduce the requirements on storage and transportation conditions of certain special reagents.

The sensitivity of some emerging detection methods (such as RAA or RPA) is subject to the addition amount of templates in the system, and because of the aqueous reconstitution procedure in the former reaction system, the adjustable range of the amount of the templates added into the system is narrow. Addition of templates regardless of the possible concentration change of the effective components in the system can easily result in reduced specificity of the reaction, as the recombinase constant-temperature amplification technology belongs to a multi-enzyme reaction. Also, in-situ lyophilizing limits the flexibility of the system in application, consumables must be fixed in the production stage, and the flexibility of reagent use and the compatible model are limited.

### SUMMARY

The present disclosure provides a microsphere preparation for nucleic acid amplification, and the microsphere preparation comprises reaction microspheres obtained by lyophilizing the mixed reagents required in an amplification reaction, and each gram of the reaction microspheres comprises:

131.58-530.5 μg of DNA polymerase, 2.632-13.263 mg of single-strand binding protein, 0.9867-3.316 mg of recombinase, 0.395-1.33 mg of auxiliary protein, 0.0075-0.02 nmol of each primer, 657.89-663.13 μg of creatine kinase, 0.1-0.2 μmol of ATP, 0.0026-0.015 mmol of DTT, 0.1-0.5 mmol of phosphokinase, 0.008-0.012 μmol of each dNTP, 0.5-2.5 μmol of Tris-Ac, 0-663.13 mg of maltose and 131.58-663.13 mg of PEG. Optionally, the microsphere preparation is used in an RNA amplification system, wherein each gram of microsphere preparation further comprises 394.74-795.76 μg of reverse transcriptase.

Optionally, the microsphere preparation comprises reaction microspheres obtained by lyophilizing the mixed reagents required in an amplification reaction, wherein each gram of the reaction microspheres comprises:

460.53-464.19 μg of DNA polymerase, 0.013 nmol of each primer, 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

Optionally, each gram of reaction microspheres comprises: 460.53-464.19 μg of DNA polymerase, 0.013 nmol of each primer, 657.89-663.13 μg of creatine kinase, 0.0026-0.015 mmol of DTT, 0.1-0.5 mmol of phosphokinase, 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

Optionally, the microsphere preparation comprises 657.89-663.13 μg of reverse transcriptase.

Optionally, the recombinase is selected from at least one of the following: T4 UvsX protein, T6 UvsX protein, or Rb69 UvsX protein.

Optionally, the auxiliary protein is selected from at least one of the following: T4 UvsY protein, T6 UvsY protein, or Rb69 UvsY protein.

Optionally, the DNA polymerase is a strand-displacing DNA polymerase selected from at least one of the following: Staphylococcus aureus DNA polymerase I large fragment, Bacillus subtilis DNA polymerase I large fragment, Escherichia coli DNA polymerase I large fragment, or T4 bacteriophage Klewnowexo-polymerase.

Optionally, the reverse transcriptase comprises M-MLV reverse transcriptase.

Optionally, the single-strand binding protein is selected from at least one of the following: T4 GP32 protein, T6 GP32 protein, or Rb69 GP32 protein.

Optionally, the microsphere preparation further comprises a second microsphere comprising a reconstitution solvent PEG and/or a magnesium salt activator.

Optionally, each gram of the second microspheres contains 960-980 mg of PEG and/or 265.0-265.5 μmol of the magnesium salt.

The present disclosure also provides use of the microsphere preparation according to any one of the above in RPA or RAA.

The present disclosure also provides a preparation method for a microsphere preparation comprising the microsphere preparation according to any one of the above;
wherein the preparation method comprises uniformly mixing all components of the microsphere preparation, dripping the mixture into liquid nitrogen at a time interval of more than or equal to 25 s, storing the microspheres in the liquid nitrogen for more than or equal to 1 h, transferring the microspheres into a lyophilizer for lyophilizing according to a lyophilization program to obtain the microsphere preparation; and
the lyophilization program is a gradient temperature-rising lyophilizing method, and sequentially comprises a pre-freezing step, a main drying step and a final drying step.

Optionally, the pre-freezing step is performed at a temperature of less than or equal to −54° C., with a treatment period of 0.5-1 h.

Optionally, the main drying step is performed at a temperature of −27 to ˜15° C., with a treatment period of 2-6 h and a vacuum degree of 0.01-30 Pa; further optionally, the main drying step comprises at least two gradient temperature-rising treatment processes.

Optionally, the final drying step is performed at a temperature of 0-20° C., with a treatment period of more than or equal to 2 h and a vacuum degree of 0.01-1 Pa. Optionally, the final drying step comprises at least four gradient temperature-rising treatment processes.

The present disclosure also provides a method for amplifying nucleic acid using the microsphere preparation according to any one of the above, and the method comprises adding reaction microspheres into an amplification sample solution according to an addition ratio of 0.263-6.58 mL of an amplification sample solution to each gram of the reaction microspheres, and then performing amplification according to any one of the following procedures (a)-(c):
(a) adding an aqueous reconstitution solvent and an activator, mixing uniformly, and directly amplifying for 20 min at 37-44° C.;
(b) after adding a second microsphere, amplifying for 20 min at 37-44° C., wherein the second microsphere comprises a reconstitution solvent PEG and a magnesium salt activator;
(c) adding a second microsphere after reconstitution, and amplifying for 20 min at 37-44° C., wherein the second microsphere comprises a magnesium salt activator.

The present disclosure also provides use of a microsphere preparation in nucleic acid amplification in combination with a second reaction method, and the microsphere preparation comprises the microsphere preparation according to any one of the above;
wherein the nucleic acid amplification adopts the aforementioned nucleic acid amplification methods;
all preparations used in the second reaction are microsphere preparations; and
the second reaction comprises a fluorescence reaction or a CRISPR reaction.

Optionally, the fluorescence reaction comprises adding an exonuclease and a probe into the RPA or RAA system prior to lyophilizing, so that the RPA or RAA fluorescence reaction can be detected in real time.

Optionally, the exonuclease is selected from exonuclease III.

Optionally, the CRISPR reaction comprises Cas12 CRISPR detection system and Cas13 CRISPR detection system.

Optionally, the second reaction method comprises adding a microsphere preparation for the second reaction directly into the amplification product to complete the second reaction.

Optionally, the second reaction is a CRISPR reaction, and the preparation method for the microsphere preparation used in the CRISPR reaction comprises: preparing a CRISPR lyophilizing system, and then preparing a microsphere preparation used for CRISPR reaction according to the aforementioned method;
wherein the CRISPR lyophilizing system comprises: 10 μL/test of CRISPR lyoprotectant, 1× Buffer, 40-100 nmol/L of Cas12 protein, 40-100 nmol/L of Cas13 protein, 5 U of mRNase inhibitor, 14 U of T7 RNA polymerase, 0.5-0.6 mM of rNTP, 0.1 μM of CrRNA1, 0.8-1.2 nmol/L of ssDNA and 0.8-1.2 nmol/L of ssRNA.

Optionally, the Cas12 protein is selected from at least one of LbCas12a, FnCas12a, AsCas12a (cpf1), BbCas12a (cpf1), and HkCas12a (cpf1).

Optionally, the Cas13 protein comprises LwaCas13a.

Optionally, the T7 RNA polymerase is derived from the expression of Escherichia coli.

The present disclosure also provides use of the microsphere preparations according to any one of the above embodiments in amplification auto-chromogenic reaction, and each gram of the microsphere preparation further comprises 0.4613-12.13 mg of an exonuclease.

Optionally, each gram of microsphere preparation comprises 0.6579-0.6667 mg of the exonuclease.

The present disclosure also provides use of the microsphere preparation according to any one of the above in the detection of a virus infection.

Optionally, the virus comprises at least one of the following: respiratory syncytial virus, influenza A virus, or influenza B virus.

### BRIEF DESCRIPTION OF DRAWINGS

In order to more clearly illustrate the specific embodiments of the present disclosure or technical solutions in the prior art, the drawings used in the descriptions of the specific embodiments or the prior art are briefly introduced below. It is obvious that the drawings in the following description are only illustrative of some embodiments of the present disclosure. On the basis of these drawings, other drawings can be obtained by those of ordinary skill in the art without creative efforts.

FIG. 1 shows a packaged form of the microsphere preparation provided by the present disclosure being used only for RPA or RAA reactions.

FIG. 2 shows a packaged form of the microsphere preparation provided by the present disclosure comprising reconstitution microspheres or activator microspheres.

FIG. 3 shows a packaged form of the microsphere preparation provided by the present disclosure being used for RPA-CRISPR reactions.

FIG. 4 shows an individually packaged form of different types of microsphere preparations provided by the present disclosure.

### DETAILED DESCRIPTION

In order to make the objectives, technical solutions and advantages of the embodiments of the present disclosure clearer, the technical solutions in the embodiments of the present disclosure will be clearly and completely described below with reference to the drawings in the embodiments of the present disclosure, and it is obvious that the described embodiments are some but not all embodiments of the present disclosure.

Thus, the following detailed description of the embodiments of the present disclosure provided in the drawings is not intended to limit the scope of the present disclosure as claimed, but is merely representative of selected embodiments of the present disclosure. Based on the embodiments of the present disclosure, all other embodiments obtained by those of ordinary skill in the art without creative efforts shall fall within the claimed scope of the present disclosure.

In the description of the present disclosure, it should be noted that the terms “first”, “second”, and the like are used only for distinguishing the description, and are not intended to indicate or imply relative importance.

An embodiment of the present disclosure provides a microsphere preparation for nucleic acid amplification, the microsphere preparation comprises reaction microspheres obtained by lyophilizing the mixed reagents required in an amplification reaction, and each gram of the reaction microspheres comprises:

131.58-530.5 μg of DNA polymerase, 2.632-13.263 mg of single-strand binding protein, 0.9867-3.316 mg of recombinase, 0.395-1.33 mg of auxiliary protein, 0.0075-0.02 nmol of each primer (namely 0.0075-0.02 nmol per single primer or 0.0075-0.02 nmol of each primer in the system), 657.89-663.13 μg of creatine kinase, 0.1-0.2 μmol of ATP, 0.0026-0.015 mmol of DTT, 0.1-0.5 mmol of phosphokinase, 0.008-0.012 μmol of each dNTP, 0.5-2.5 μmol of Tris-Ac, 0-663.13 mg of maltose and 131.58-663.13 mg of PEG.

In an optional embodiment, an amount of the DNA polymerase can be, for example, 131.60-530.0 μg, 300.0-500.0 μg, or 450.0-480.0 μg, such as 131.58 μg, 200 μg, 300 μg, 380 μg, 400 μg, 450 ug, 460 μg, 470 μg, 500 μg, 530 μg, or 530.5 μg, or a value in the interval between any two endpoints. The amount of single-strand binding protein can be, for example, 2.70-13.0 mg, 8.0-12.0 mg or 9.0-11.0 mg, such as 2.632 mg, 5.0 mg, 7.0 mg, 9.0 mg, 10.0 mg, 11.0 mg or 13.0 mg, or a value in the interval between any two endpoints. The amount of recombinase can be, for example, 0.99-3.30 mg, 1.0-3.0 mg, 1.5-2.5 mg, or 1.5-2.0 mg, such as 0.99 mg, 1.0 mg, 1.5 mg, 1.7 mg, 1.75 mg, 1.8 mg, 2.0 mg, 2.5 mg, 3.0 mg, or 3.3 mg, or a value in the interval between any two endpoints. The amount of auxiliary protein can be, for example, 0.40-1.30 mg, 0.80-1.20 mg or 0.90-1.10 mg, such as 0.5 mg, 0.8 mg, 0.9 mg, 1.0 mg, 1.1 mg or 1.3 mg, or a value in the interval between any two endpoints. The amount of each primer in the system can be, for example, 0.008-0.018 nmol, 0.0095-0.016 nmol, or 0.010-0.014 nmol, such as 0.0080 nmol, 0.0095 nmol, 0.010 nmol, 0.012 nmol, 0.013 nmol, 0.014 nmol, 0.016 nmol, or 0.018 nmol, or a value in the interval between any two endpoints. The amount of creatine kinase can be, for example, 657.90-663.00 μg, 658.0-662.0 μg, or 659.5-661.5μ, such as 657.90μ, 658.0μ, 659.0μ, 660.0μ, 661.0μ, 662.0μ, or 663.0 μg, or a value in the interval between any two endpoints. The amount of ATP can be, for example, 0.1-0.2 μmol, such as 0.1 μmol, 0.12 μmol, 0.14 μmol, 0.15 μmol, 0.16 μmol, 0.18 μmol or 0.2 μmol, or a value in the interval between any two endpoints. The amount of DTT can be, for example, 0.0030-0.0140 mmol, 0.0080-0.0138 mmol, or 0.010-0.0135 mmol, such as 0.0030 mmol, 0.0070 mmol, 0.0090 mmol, 0.010 mmol, 0.012 mmol, 0.013 mmol, 0.014 mmol, or 0.015 mmol, or a value in the interval between any two endpoints. The amount of phosphokinase can be, for example, 0.15-0.45 mmol, 0.20-0.40 mmol or 0.25-0.35 mmol, such as 0.1 mmol, 0.18 mmol, 0.20 mmol, 0.28 mmol, 0.32 mmol, 0.33 mmol, 0.34 mmol, 0.36 mmol, 0.38 mmol, 0.40 mmol, 0.46 mmol or 0.5 mmol, or a value in the interval between any two endpoints. The amount of dNTP can be, for example, 0.0085-0.018 μmol, 0.0090-0.016 μmol, or 0.0095-0.015 μmol, such as 0.008 μmol, 0.009 μmol, 0.010 μmol, 0.011 μmol, or 0.012 μmol, or a value in the interval between any two endpoints. The amount of Tris-Ac can be, for example, 0.5-2.0 μmol, 0.7-1.5 μmol, or 0.8-1.0 μmol, such as 0.5 μmol, 0.7 μmol, 0.9 μmol, 1.0 μmol, 1.5 μmol, 2.0 μmol, or 2.5 μmol, or a value in the interval between any two endpoints. The amount of maltose can be, for example, 0.1-660 mg, 50-600 mg or 150-500 mg, such as 0.1 mg, 10 mg, 100 mg, 150 mg, 200 mg, 250 mg, 300 mg, 350 mg, 370 mg, 390 mg, 400 mg, 450 mg, 500 mg, 550 mg, 600 mg, 650 mg or 663 mg, or a value in the interval between any two endpoints. The amount of maltose can be 0, i.e., no maltose is added. The amount of PEG can be, for example, 135-600 mg, 140-450 mg or 145-185 mg, such as 131.58 mg, 135 mg, 140 mg, 145 mg, 150 mg, 160 mg, 170 mg, 180 mg, 190 mg, 200 mg, 300 mg, 500 mg, 600 mg or 663.13 mg, or a value in the interval between any two endpoints.

In an optional embodiment, the microsphere preparation is used in an RNA amplification system, and each gram of microsphere preparation further comprises 394.74-795.76 μg of reverse transcriptase. For example, the amount of the reverse transcriptase can be 395.0-795.0μ, 450.0-750.0 μg or 550.0-650.0μ, such as 394.74μ g, 395.0 μg, 400.0 μg, 450 μg, 500 μg, 550 μg, 600 μg, 650 μg, 700 μg, 750 μg or 790 μg, or a value in the interval between any two endpoints.

In an optional embodiment, the microsphere preparation comprises reaction microspheres obtained by lyophilizing the mixed reagents required in an amplification reaction, and each gram of the reaction microspheres comprises:

460.53-464.19 μg of DNA polymerase, 0.013 nmol of each primer (namely 0.013 nmol per single primer or 0.013 nmol of each primer in the system), 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

In an optional embodiment, each gram of reaction microspheres comprises: 460.53-464.19 μg of DNA polymerase, 0.013 nmol of each primer (namely 0.013 nmol per single primer or 0.013 nmol of each primer in the system), 657.89-663.13 μg of creatine kinase, 0.0026-0.015 mmol of DTT, 0.1-0.5 mmol of phosphokinase, 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

Optionally, the microsphere preparation further comprises 657.89-663.13 μg of reverse transcriptase.

In an optional embodiment, the recombinase is selected from T4 UvsX protein, T6 UvsX protein, and Rb69 UvsX protein. In an optional embodiment, the recombinase is selected from at least one of T4 UvsX protein, T6 UvsX protein, and Rb69 UvsX protein.

Optionally, the auxiliary protein is selected from T4 UvsY protein, T6 UvsY protein and Rb69 UvsY protein. Optionally, the auxiliary protein is selected from at least one of T4 UvsY protein, T6 UvsY protein and Rb69 UvsY protein.

Optionally, the DNA polymerase is a strand-displacing DNA polymerase selected from Staphylococcus aureus DNA polymerase I large fragment, Bacillus subtilis DNA polymerase I large fragment, Escherichia coli DNA polymerase I large fragment, and T4 bacteriophage Klewnowexo-polymerase. Optionally, the DNA polymerase is a strand-displacing DNA polymerase selected from at least one of Staphylococcus aureus DNA polymerase I large fragment, Bacillus subtilis DNA polymerase I large fragment, Escherichia coli DNA polymerase I large fragment, and T4 bacteriophage Klewnowexo-polymerase.

Optionally, the reverse transcriptase comprises M-MLV reverse transcriptase.

Optionally, the single-strand binding protein is selected from T4 GP32 protein, T6 GP32 protein, and Rb69 GP32 protein. Optionally, the single-strand binding protein is selected from at least one of T4 GP32 protein, T6 GP32 protein, and Rb69 GP32 protein.

In an optional embodiment, the microsphere preparation further comprises a second microsphere comprising a reconstitution solvent PEG and/or a magnesium salt activator.

Optionally, each gram of the second microspheres contains 960-980 mg of PEG and/or 265.0-265.5 μmol of magnesium salt.

An embodiment of the present disclosure provides use of the microsphere preparation according to any one of the above embodiments in RPA or RAA.

An embodiment of the present disclosure provides a preparation method for a microsphere preparation, and the microsphere preparation comprises the microsphere preparation according to any one of the above embodiments;
the preparation method comprises uniformly mixing all components of the microsphere preparation, dripping the mixture into liquid nitrogen at a time interval of more than or equal to 25 s, storing the microspheres in the liquid nitrogen for more than or equal to 1 h, transferring the microspheres into a lyophilizer for lyophilizing according to a lyophilization program to obtain the microsphere preparation;
the lyophilization program is a gradient temperature-rising lyophilizing method, and sequentially comprises a pre-freezing step, a main drying step and a final drying step.

Optionally, the pre-freezing step is performed at a temperature of less than or equal to −54° C., with a treatment period of 0.5-1 h.

Optionally, the main drying step is performed at a temperature of −27 to ˜15° C., with a treatment period of 2-6 h and a vacuum degree of 0.01-30 Pa. Optionally, the main drying step comprises at least two gradient temperature-rising treatment processes.

Optionally, the final drying step is performed at a temperature of 0-20° C., with a treatment period of more than or equal to 2 h and a vacuum degree of 0.01-1 Pa. Optionally, the final drying step comprises at least four gradient temperature-rising treatment processes.

An embodiment of the present disclosure also provides a method for amplifying nucleic acid using the microsphere preparation according to any one of the above embodiments, and the method comprises adding reaction microspheres into an amplification sample solution according to an addition ratio of 0.263-6.58 mL of an amplification sample solution to each gram of reaction microspheres, and then performing amplification according to any one of the following procedures (a)-(c):
(a) adding an aqueous reconstitution solvent and an activator, mixing uniformly, and directly amplifying for 20 min at 37-44° C.;
(b) after adding a second microsphere, amplifying for 20 min at 37-44° C., wherein the second microsphere comprises a reconstitution solvent PEG and a magnesium salt activator;
(c) adding a second microsphere after reconstitution, and amplifying for 20 min at 37-44° C., wherein the second microsphere comprises a magnesium salt activator.

The present disclosure provides use of a microsphere preparation in nucleic acid amplification in combination with a second reaction method, and the microsphere preparation comprises the microsphere preparation according to any one of the above embodiments;
the nucleic acid amplification adopts the aforementioned nucleic acid amplification methods;
all preparations used in the second reaction are microsphere preparations;
the second reaction comprises a fluorescence reaction or a CRISPR reaction.

Optionally, the fluorescence reaction comprises adding an exonuclease and a probe into the RPA or RAA system prior to lyophilizing, so that the RPA or RAA fluorescence reaction can be detected in real time.

Optionally, the exonuclease is selected from exonuclease III.

Optionally, the CRISPR reaction comprises Cas12 CRISPR detection system and Cas13 CRISPR detection system.

In an optional embodiment, the second reaction method comprises adding a microsphere preparation for the second reaction directly into the amplification product to complete the second reaction.

In an optional embodiment, the second reaction is a CRISPR reaction, and the preparation method for the microsphere preparation used in the CRISPR reaction comprises: preparing a CRISPR lyophilizing system, and then preparing a microsphere preparation used for CRISPR reaction according to the methods used in the aforementioned embodiments;
the CRISPR lyophilizing system comprises: 10 μL/test of CRISPR lyoprotectant, 1×Buffer, 40-100 nmol/L of Cas12 protein, 40-100 nmol/L of Cas13 protein, 5 U of mRNase inhibitor, 14 U of T7 RNA polymerase, 0.5-0.6 mM of rNTP, 0.1 μM of CrRNA1, 0.8-1.2 nmol/L of ssDNA and 0.8-1.2 nmol/L of ssRNA.

Optionally, the Cas12 protein is selected from LbCas12a, FnCas12a, AsCas12a (cpf1), BbCas12a (cpf1), and HkCas12a (cpf1). Optionally, the Cas12 protein is selected from at least one of LbCas12a, FnCas12a, AsCas12a (cpf1), BbCas12a (cpf1), and HkCas12a (cpf1).

Optionally, the Cas 13 protein comprises LwaCas13a.

Optionally, the T7 RNA polymerase is derived from the expression of Escherichia coli.

An embodiment of the present disclosure also provides use of the microsphere preparation according to any one of the above embodiments in amplification auto-chromogenic reaction, and each gram of the microsphere preparation further comprises 0.4613-12.13 mg of an exonuclease.

Optionally, each gram of microsphere preparation comprises 0.6579-0.6667 mg of the exonuclease.

An embodiment of the present disclosure further provides use of the microsphere preparation according to any one of the above embodiments in the detection of a virus infection.

Optionally, the virus comprises at least one of respiratory syncytial virus, influenza A virus, and influenza B virus.

Firstly, the specific different reaction methods related to in the specific embodiment of the present disclosure are listed as follows:

### 1. Lyophilizing Preparation Method for Microsphere Preparation

In some specific embodiments below, the lyophilizing method used to prepare the microsphere preparation is uniformly mixing all components of the microsphere preparation, then dripping the mixture into liquid nitrogen at a time interval of more than or equal to 25 s (the upper limit of the time interval is not limited, any time interval more than or equal to 25 s is applicable to this method), storing the microspheres in the liquid nitrogen for more than or equal to 1 h (the upper limit of the storage time is not limited, any storage time more than or equal to 1 h is applicable to this method), and transferring the microspheres into a lyophilizer for lyophilizing according to a lyophilization program to obtain the microsphere preparation. The lyophilization program is a gradient temperature-rising lyophilizing method, and sequentially comprises a pre-freezing step, a main drying step and a final drying step. The pre-freezing step is performed at a temperature of less than or equal to −54° C., with a treatment period of 0.5-1 h; the main drying step is performed at a temperature of −27 to −15° C., with a treatment period of 2-6 h and a vacuum degree of 0.01-30 Pa; the main drying step comprises at least two gradient temperature-rising treatment processes; the final drying step is performed at a temperature of 0-20° C., with a treatment period of more than or equal to 2 h and a vacuum degree of 0.01-1 Pa; and the final drying step comprises at least four gradient temperature-rising treatment processes.

### 2. RAA and RPA (Basic and Fluorescent) Reaction Parameters

The compositions of the microspheres used in the RAA and RPA (basic and fluorescent) reactions are as described above and with variation between them. However, the specific reaction methods can follow the same steps, including but not limited to mixing the reaction system, then keeping the temperature at 37-44° C. for 20 min, and if the reaction is fluorescent, for 40 cycles in total, acquiring fluorescence every 30 s according to the fluorescence acquisition rule.

### 3. CRISPR Reaction

In some specific embodiments below, the CRISPR reaction comprises, but is not limited to the steps of mixing 10 μL of the inactivated amplification product with 25 μL of water, adding the mixture to the CRISPR microspheres, performing the reaction at 45-65° C. with ABI7500, and acquiring fluorescence every 30 s for 30 times in total.

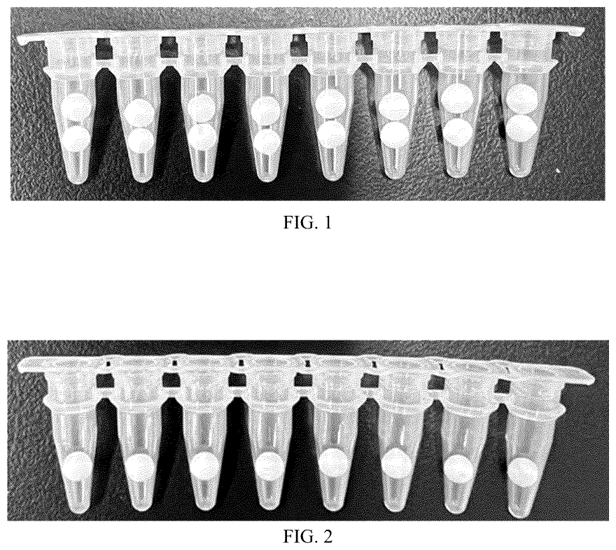

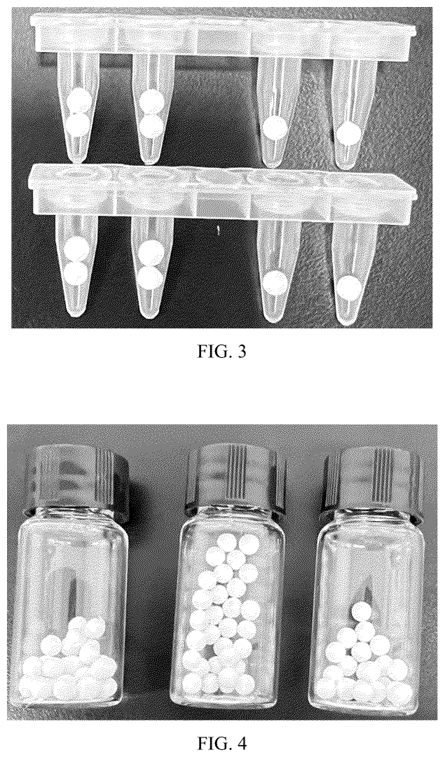

Secondly, the specific packaged forms of the microsphere preparations for different uses provided by the present disclosure are as follows:
1. The microsphere preparation used only for RPA or RAA reaction is shown in FIG. 1, wherein the amplification reagents including the required primers are subjected to the lyophilizing preparation method of the microsphere preparation to obtain independent microspheres, and each individual microsphere is independently packaged; when performing RPA or RAA reaction, a sample to be detected, an aqueous reconstitution solvent and an activator are directly added.
2. If some reactions also need the addition of a reconstitution microsphere, the packaged form shown in FIG. 2 is correspondingly adopted. Each package tube contains a reaction microsphere and a reconstitution microsphere, and the sample to be detected and an aqueous activator are directly added when performing the reaction. It is understood that the aqueous magnesium salt activator may also be mixed with a reconstitution solvent and lyophilized into a reconstitution/activation mixed microsphere, and then the sample to be tested can be directly added.
3. If the completed RPA or RAA reaction is combined with a second reaction, such as a CRISPR reaction, the corresponding packaged form is shown in FIG. 3, wherein the two left side tubes are package tubes for completing RPA or RAA reaction, the two right side tubes contain reaction microspheres for CRISPR reaction, and the products after the amplification from the left side package tubes are directly transferred into the right side package tubes to perform CRISPR reaction.

It should be noted that the package tubes used in the above three packaged forms can be adjusted and replaced according to circumstance requirements, and are not limited to the specific structures and shapes of the package tubes in the drawings.
4. For bulk storage, transportation and sale without the need of immediate use, different types of microsphere preparations can also be packaged separately, as shown in FIG. 4.

The present disclosure provides a microsphere preparation, wherein in the amplification reaction process, an aqueous sample to be detected is directly mixed with the microsphere preparation, and the preparation step of the amplification reagents is omitted, so that the concentration of the primers is always in a stable supersaturated state, therefore the sensitivity of the multiple detection reaction is improved, while the specificity of the amplification reaction is not negatively affected.

The microsphere preparation for nucleic acid amplification provided by the present disclosure can be stored for a long period at 2-8° C., and when being used, without necessarily adding an extra solvent, the microsphere preparation is directly mixed with a sample to be detected, so that the upper limit of the concentration of a template can be significantly improved, and therefore the sensitivity in multiple detection is enhanced.

The use of the microsphere preparation for nucleic acid amplification provided by the present disclosure for RPA and RAA, or for a dual or multiple detection formed on the basis of coupling RPA or RAA with a second reaction can improve the sensitivity significantly, and meanwhile ensure the amplification specificity.

### EXAMPLES

Some embodiments of the present disclosure are described in detail below with reference to the attached drawings. The examples and features of the examples described below can be combined with each other without conflict.

In the following examples, the detection rate is calculated by the formula: detection rate=number of samples detected/total number of samples detected.

### Example 1

This example provides lyophilized RPA reaction microspheres for respiratory syncytial virus typing assay, and each gram of the reaction microspheres comprises the following components as shown in Tables 1 and 2 below:

TABLE 1

| Component name | Content | |
|---|---|---|
| T4 GP32 | 10.53-10.61 | mg |
| T4 UvsX | 1.71-1.72 | mg |
| T4 UvsY | 1.05-1.06 | mg |
| Klewnowexo-polymerase | 460.53-464.19 | μg |
| M-MLV | 657.89-663.13 | μg |
| Exonuclease III | 0.6579-0.6667 | mg |
| Maltose | 394.74-397.88 | mg |
| PEG (20K-36K) | 150-151.19 | mg |
| Creatine kinase | 657.89-663.13 | μg |
| ATP | 0.15 | μmol |
| DTT | 0.013 | mmol |
| Phosphokinase | 0.331 | mmol |
| dNTPs (i.e., including dATP, dGTP, dTTP, | 0.01 μmol each | |
| and dCTP) | | |
| Tris-Ac | 0.5 | μmol |

| | |
|---|---|
| Primers (in Table 2) | 0.013 nmol each |
| Probe | 0.006 nmol each |

The primers are detailed in Table 2:

TABLE 2

| Name | Sequence (5′-3′) |
|---|---|
| RSV-P | CCGTTGGATGGTGTATTTGCTGGATGACAGAAGTTNATCT |
| | TTGTTGAGTG (SEQ ID No: 1) |
| RSV-F | ATGGCTCTTAGCAAAGTCAAGTTGAATGATACA |
| | (SEQ ID No: 2) |
| RSV-R | GTTTCTGCACATCATAATTAGGAGTATCAATA |
| | (SEQ ID No: 3) |
| RSV-P1 | TCTCCTGTACTACGTTGAATAGTGTATNTGCTGGATGACA |
| | GCAGC (SEQ ID No: 4) |
| RSV-2R | GCACATCATAATTAGGAGTATCAATA |
| | (SEQ ID No: 5) |

The combination of RSV-F with RSV-R and RSV-P can specifically detect respiratory syncytial virus type A; the combination of RSV-F with RSV-2R and RSV-P1 can specifically detect respiratory syncytial virus type B. The 35th and the 38th base T of the RSV-P nucleotide sequence were coupled with FAM and BHQ1 fluorescent groups, respectively, and the 36th base was replaced by THF. The 27th and the 29th base T of the RSV-P1 nucleotide sequence were coupled with CY5 and BHQ2 fluorescent groups, respectively, and the 28th base was replaced by THF.

It should be noted that the microsphere preparation provided by the present disclosure is not selective for but well compatible with various primers and probes, and those skilled in the art can independently design corresponding primers and probes according to actual amplification targets.

A white microsphere preparation was prepared according to the microsphere lyophilizing preparation method aforementioned, and the specific lyophilization program parameters are as follows in Table 3 below:

TABLE 3

| | Procedure | | | | | | |
|---|---|---|---|---|---|---|---|
| | Pre-freezing | Main drying | | Final drying | | | |
| | Time (h) | | | | | | |
| | 1 | 6 | 2 | 2 | 2 | 2 | — |
| Shelf | −54 | −27 | −15 | 0 | 10 | 15 | 20 |
| temperature (° C.) | | | | | | | |
| Set vacuum | / | 0.1 | 0.1 | 0.1 | 0.1 | 0.1 | 0.1 |
| degree (Pa) | | | | | | | |

The obtained reaction microspheres were about 7.54-7.60 mg in weight, 3.8-4.5 mm in diameter, white and spherical.

Respiratory syncytial type a/b oropharyngeal swab samples (sample source: provided by Shanghai BioGerm Medical Testing Laboratory) quantified by digital PCR were diluted to 1×10⁴ copies/mL, 5×10³ copies/mL, 5×10² copies/mL, 2.5×10² copies/mL and 1.5×10² copies/mL as samples to be tested for later use. The aforementioned reaction microspheres prepared were used for detecting each sample repeatedly for 10 times, and the sample with the detection rate reaching 90% indicates the lowest detection limit of the preparation.

### Detection Flow:

(1) Reaction microspheres were aliquoted into reaction tubes in advance, 1 RPA reaction microsphere per well in the RPA reaction tube.
(2) 15 μL of the sample was added to the RPA reaction microspheres, and 30 μL of reconstitution solvent and 5 μL of activator were added; the reconstitution solvent was a 7.35% (w/v) PEG (35K) aqueous solution, and the activator was a 140 mM magnesium acetate aqueous solution.
(3) After the reaction system was mixed uniformly, the reaction was performed at 44° C. with ABI7500, fluorescence was acquired every 30 s for 30 times in total, and the corresponding detection rate was statistically analyzed.

The results are detailed in Table 4:

TABLE 4

| | | Positive | Positive | |
|---|---|---|---|---|
| | | detection | detection | Negative |
| | | rate of | rate of | detection |
| | | respiratory | respiratory | rate of |
| Template | | syncytial | syncytial | negative |
| concentration | | virus type A | virus type B | control |
| 1 × 10⁴ | copies/mL | 10/10 | 10/10 | 3/3 |
| 5 × 10³ | copies/mL | 10/10 | 10/10 | 3/3 |
| 5 × 10² | copies/mL | 10/10 | 10/10 | 3/3 |
| 2.5 × 10² | copies/mL | 10/10 | 10/10 | 3/3 |
| 1.5 × 10² | copies/mL | 6/10 | 7/10 | 3/3 |

Conclusion: the dual system microspheres can detect different types of respiratory syncytial samples, and the detection sensitivity is as low as 2.5×10² copies/mL.

### Example 2

This example differs only from Example 1 in that reconstitution microspheres with a component of PEG (35K) (976 mg/g) were also prepared using the lyophilizing preparation method.

Respiratory syncytial type a/b oropharyngeal swab samples (sample source: provided by Shanghai BioGerm Medical Testing Laboratory) quantified by digital PCR were diluted to 5×10³ copies/mL, 5×10² copies/mL, 2.5×10² copies/mL, 1.5×10² copies/mL and 1.0×10² copies/mL as samples to be tested for later use. The rest of the tests were the same as in Example 1, and the results are shown in Table 5 below:

TABLE 5

| | | Positive | Positive | |
|---|---|---|---|---|
| | | detection | detection | Negative |
| | | rate of | rate of | detection |
| | | respiratory | respiratory | rate of |
| Template | | syncytial | syncytial | negative |
| concentration | | virus type A | virus type B | control |
| 5 × 10³ | copies/mL | 10/10 | 10/10 | 3/3 |
| 5 × 10² | copies/mL | 10/10 | 10/10 | 3/3 |
| 2.5 × 10² | copies/mL | 10/10 | 10/10 | 3/3 |
| 1.5 × 10² | copies/mL | 10/10 | 10/10 | 3/3 |
| 1.0 × 10² | copies/mL | 8/10 | 8/10 | 3/3 |

Conclusion: the dual system microspheres can detect different types of respiratory syncytial samples, and the detection sensitivity is as low as 1.5×10² copies/mL.

### Example 3

This example provides RPA-CRISPR lyophilized microspheres for the detection of influenza A and influenza B viruses, wherein the microspheres comprise RPA reaction microspheres, reconstitution microspheres, and CRISPR microspheres. The reaction microspheres were about 7.54-7.60 mg in weight, 3.8-4.5 mm in diameter, white and spherical; the reconstitution microspheres were about 2.52-2.58 mg in weight, 3.2-3.5 mm in diameter, white and spherical; and the CRISPR microspheres were about 2.2-2.32 mg in weight, 3.2-3.8 mm in diameter, light pink and spherical. The components of the RPA reaction microspheres per gram are shown in Table 6 below:

TABLE 6

| | |
|---|---|
| Component name | Content |

| Single-strand binding protein | 10.53-10.61 | mg |
|---|---|---|
| Recombinase | 1.71-1.72 | mg |
| Auxiliary protein | 1.05-1.06 | mg |
| DNA polymerase | 460.53-464.19 | μg |
| Reverse transcriptase | 657.89-663.13 | μg |
| Maltose | 394.74-397.88 | mg |
| PEG (20K-36K) | 150-151.19 | mg |
| Creatine kinase | 657.89-663.13 | μg |
| ATP | 0.15 | μmol |
| DTT | 0.013 | mmol |
| Phosphokinase | 0.331 | mmol |
| dNTPs (i.e., including dATP, dGTP, | 0.01 μmol each | |
| dTTP, and dCTP) | | |
| Tris-Ac | 0.5 | μmol |

| | |
|---|---|
| Primers (in Table 7) | 0.015 nmol each |

The primers are detailed in Table 7:

TABLE 7

| Name | Sequence (5′-3′) |
|---|---|
| IFA-F | TAATACGACTCACTATAGGGATGAGYCTTCTAACYGARGT |
| | CGAAACGTACGTTC (SEQ ID No: 6) |
| IFA-R | CGTCTACGCTGCAGTCCYCGCTCACTGGGC |
| | (SEQ ID No: 7) |
| IFB-F | TTACACTGTTGGTTYGGTGGGAAAGAATTTGACCT |
| | (SEQ ID No: 8) |
| IFB-R | TTCTTTTTTGTTGCTGTTGTYCCCATTCCYGA |
| | (SEQ ID No: 9) |

IFA-F and IFA-R can be used in combination to specifically amplify the target sequence of influenza A virus, and IFB-F and IFB-R can be used in combination to specifically amplify the target sequence of influenza B virus.

The component of the reconstitution microspheres was PEG (35K): 976 mg/g.

CRISPR microsphere lyophilizing system is as follows in Table 8 below:

TABLE 8

| Component name | Addition amount | |
|---|---|---|
| CRISPR lyoprotectant | 10 | μL/test |
| Buffer 3.1 | 1× | |
| LbCas12a(cpf1) | 100 | nmol/L |
| LwaCas13a | 100 | nmol/L |
| Murine RNase inhibitor (40 U/uL) | 0.125 | μL |
| T7 RNA polymerase | 0.05 | μL |
| rNTP | 0.8 | μL |
| CrRNA1 | 20 | ng/μL |
| CrRNA2 | 20 | ng/μL |
| ssDNA | 2 | μmol/L |
| ssRNA | 2 | μmol/L |

The nucleotide sequences of CrRNA1, CrRNA2, ssDNA and ssRNA are shown in Table 9 below:

TABLE 9

| Name | Sequence (5′-3′) |
|---|---|
| CrRNA1 | GAUUUAGACUACCCCAAAAACGAAGGGGACUAAAACGAC |
| | AAGACCAAUCCUGUCACCUCUGACU (SEQ ID |
| | No: 10) |
| CrRNA2 | UAAUUUCUACUAAGUGUAGAUGUAUAUCAGUUAAGCAUC |
| | UUUUG (SEQ ID No: 11) |
| SSDNA | 5′6-FAM-TTATT-3′-BHQ1 (SEQ ID No: 12) |
| SSRNA | 5′6-VIC-UUUUU-3′-BHQ1 (SEQ ID No: 13) |

The CrRNA1 can specifically bind to the amplification products of IFA-F and IFA-R in the CRISPR system to initiate the trans-cleavage activity of Cas13 protein to cleave the ssRNA and emit fluorescence; the CrRNA2 can specifically bind to the amplification products of IFB-F and IFB-R in the CRISPR system to initiate the trans-cleavage activity of Cas12 protein to cleave the ssDNA and emit fluorescence.

Influenza A and influenza B virus oropharyngeal swab samples (sample source: provided by Shanghai BioGerm Medical Testing Laboratory) quantified by digital PCR were diluted to 1×10⁴ copies/mL, 5×10³ copies/mL, 5×10² copies/mL, 2.5×10² copies/mL, 2.0×10² copies/mL and 1.0×10² copies/mL as samples to be tested for later use.

The detection flow was as follows:
(1) Reaction microspheres were aliquoted into reaction tubes in advance, 1 RPA reaction microsphere and 1 reconstitution solvent microsphere per well in the RPA reaction tube; 1 CRISPR reaction microsphere per well in the CRISPR reaction tube.
(2) 45 μL of the sample was added to the RPA reaction microspheres, and 5 μL of activator was added.
(3) After the reaction system was mixed uniformly, the mixture was incubated in a metal bath at 42° C. for 20 min and then inactivated at 95° C. for 2 min.
(4) 10 μL of the inactivated RPA product was mixed with 25 L of water uniformly, and the mixture was added to the CRISPR microspheres.
(5) The reaction was performed at 45° C. with ABI7500, fluorescence was acquired every 30 s for 30 times in total, and the corresponding detection rate was statistically analyzed.

The results are shown in Table 10 below:

TABLE 10

| | | | | Negative |
|---|---|---|---|---|
| | | Positive | Positive | detection |
| | | detection | detection | rate of |
| | | rate of | rate of | negative |
| Template concentration | | influenza A | influenza B | control |
| 1 × 10⁴ | copies/mL | 10/10 | 10/10 | 3/3 |
| 5 × 10³ | copies/mL | 10/10 | 10/10 | 3/3 |
| 5 × 10² | copies/mL | 10/10 | 10/10 | 3/3 |
| 2.5 × 10² | copies/mL | 10/10 | 10/10 | 3/3 |
| 2.0 × 10² | copies/mL | 10/10 | 10/10 | 3/3 |
| 1.0 × 10² | copies/mL | 6/10 | 8/10 | 3/3 |

Conclusion: the lowest detection limit of lyophilized RPA-CRISPR microspheres for influenza A/B dual detection was tested to be 2.0×10² copies/mL.

### Example 4

This example differs from Example 3 in that the reconstitution microspheres were omitted, an extra 30 μL of reconstitution solvent was added to the system in step (2) of the detection flow, and the samples to be tested were diluted to concentrations of 1×10⁴ copies/mL, 5×10³ copies/mL, 5×10² copies/mL, 4×10² copies/mL, 2.0×10² copies/mL and 1.0×10² copies/mL.

The detection results are shown in Table 11 below:

TABLE 11

| | | Positive | Positive | Negative |
|---|---|---|---|---|
| | | detection | detection | detection rate |
| | | rate of | rate of | of negative |
| Template concentration | | influenza A | influenza B | control |
| 1 × 10⁴ | copies/mL | 10/10 | 10/10 | 3/3 |
| 5 × 10³ | copies/mL | 10/10 | 10/10 | 3/3 |
| 5 × 10² | copies/mL | 10/10 | 10/10 | 3/3 |
| 4.0 × 10² | copies/mL | 10/10 | 10/10 | 3/3 |
| 2.0 × 10² | copies/mL | 8/10 | 9/10 | 3/3 |
| 1.0 × 10² | copies/mL | 6/10 | 8/10 | 3/3 |

Conclusion: the lowest detection limit of lyophilized RPA-CRISPR microspheres for influenza A/B dual detection was tested to be 4.0×10² copies/mL.

### Example 5

The RPA-CRISPR lyophilized microspheres for detecting influenza A and B viruses provided in Example 3 were stored at room temperature for 15 days, 30 days, 90 days, 180 days, 270 days, and 360 days separately, and then tested for stability.

Sample preparation: influenza A and B virus samples quantified by digital PCR were diluted to the lowest detection limit of 200 copies/mL as samples to be detected for later use.

The RPA-CRISPR lyophilized microspheres after different storage times were used to test the samples to be detected according to the detection method of Example 3, and the results are shown in Table 12 below:

TABLE 12

| | Storage time of reagent | | | | | |
|---|---|---|---|---|---|---|
| | 15 | 30 | 90 | 180 | 270 | 360 |
| | days | days | days | days | days | days |
| Influenza A | 10/10 | 10/10 | 10/10 | 10/10 | 10/10 | 10/10 |
| virus | | | | | | |
| Influenza B | 10/10 | 10/10 | 10/10 | 10/10 | 10/10 | 10/10 |
| virus | | | | | | |
| Negative | 3/3 | 3/3 | 3/3 | 3/3 | 3/3 | 3/3 |
| control | | | | | | |

Conclusion: the detection performance of the lyophilized microspheres after 360 days of storage at room temperature was not affected.

Finally, it should be noted that the above examples are only used to illustrate the technical solution of the present disclosure, and should not limit the same. Although the present disclosure is described in detail with reference to the examples described above, it will be appreciated by those skilled in the art that, the technical solution in the examples described above can still be modified, or some or all of the technical features can be equivalently substituted. Such modifications or substitutions do not make the technical solution corresponding thereto depart from the scope of the technical solution in the examples of the present disclosure.

### INDUSTRIAL APPLICABILITY

The microsphere preparation for nucleic acid amplification provided by the present disclosure can be stored for a long period at 2-8° C., and when being used, without necessarily adding an extra solvent, the microsphere preparation is directly mixed with a sample to be detected, so that the upper limit of the concentration of a template can be significantly improved without changing the concentration of the former system, and therefore the detection sensitivity is enhanced. The use of the microsphere preparation for nucleic acid amplification provided by the present disclosure for RPA and RAA, or for a dual or multiple detection formed on the basis of coupling RPA or RAA with a second reaction can improve the sensitivity significantly and ensure the amplification specificity, and therefore the microsphere preparation has an excellent performance in practical applications.

What is claimed is:

1. A microsphere preparation for nucleic acid amplification, comprising: a reaction microsphere obtained by lyophilizing mixed reagent required in an amplification reaction, wherein each gram of the reaction microspheres comprises:
131.58-530.5 μg of DNA polymerase, 2.632-13.263 mg of single-strand binding protein, 0.9867-3.316 mg of recombinase, 0.395-1.33 mg of auxiliary protein, 0.0075-0.02 nmol of each primer, 657.89-663.13 μg of creatine kinase, 0.1-0.2 μmol of ATP, 0.0026-0.015 mmol of DTT, 0.1-0.5 mmol of phosphokinase, 0.008-0.012 μmol of each dNTP, 0.5-2.5 μmol of Tris-Ac, 0-663.13 mg of maltose and 131.58-663.13 mg of PEG.

2. The microsphere preparation according to claim 1, wherein the microsphere preparation is used in an RNA amplification system, and each gram of the microsphere preparation further comprises 394.74-795.76 μg of reverse transcriptase.

3. The microsphere preparation according to claim 1, wherein the microsphere preparation comprises the reaction microspheres obtained by lyophilizing the mixed reagents required for an amplification reaction, and each gram of the reaction microspheres comprises:
460.53-464.19 μg of DNA polymerase, 0.013 nmol of each primer, 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

4. The microsphere preparation according to claim 3, wherein the recombinase is selected from at least one of the following: T4 UvsX protein, T6 UvsX protein and Rb69 UvsX protein:
the auxiliary protein is selected from at least one of the following: T4 UvsY protein, T6 UvsY protein, and Rb69 UvsY protein;
the DNA polymerase is a strand-displacing DNA polymerase selected from at least one of the following: Staphylococcus aureus DNA polymerase I large fragment, Bacillus subtilis DNA polymerase I large fragment, Escherichia coli DNA polymerase I large fragment, or T4 bacteriophage Klewnowexo-polymerase;
the reverse transcriptase comprises M-MLV reverse transcriptase;
the single-strand binding protein is selected from at least one of the following: T4 GP32 protein, T6 GP32 protein, or Rb69 GP32 protein.

5. The microsphere preparation according to claim 4, wherein the microsphere preparation further comprises a second microsphere, and the second microsphere comprises a reconstitution solvent PEG and/or a magnesium salt activator;
wherein each gram of the second microspheres comprises 960-980 mg of the PEG and/or 265.0-265.5 μmol of the magnesium salt.

6. (canceled)

7. A preparation method for a microsphere preparation, wherein the microsphere preparation comprises the microsphere preparation according to claim 1;
wherein the preparation method comprises uniformly mixing all components of the microsphere preparation, dripping the mixture into liquid nitrogen at a time interval of more than or equal to 25 s, storing the microspheres in the liquid nitrogen for more than or equal to 1 h, transferring the microspheres into a lyophilizer for lyophilizing according to a lyophilization program to obtain the microsphere preparation;
the lyophilization program is a gradient temperature-rising lyophilizing method, and sequentially comprises a pre-freezing step, a main drying step and a final drying step;
wherein the main drying step comprises at least two gradient temperature-rising treatment processes; and
the final drying step comprises at least four gradient temperature-rising treatment processes.

8. A method for amplifying nucleic acid using the microsphere preparation according to claim 1, wherein the method comprises adding the reaction microspheres into an amplification sample solution according to an addition ratio of 0.263-6.58 mL of the amplification sample solution to each gram of the reaction microspheres, and then performing amplification according to any one of the following procedures (a)-(c):
(a) adding an aqueous reconstitution solvent and an activator, mixing uniformly, and directly amplifying for 20 min at 37-44° C.;
(b) after adding a second microsphere, amplifying for 20 min at 37-44° C., wherein the second microsphere comprises a reconstitution solvent PEG and a magnesium salt activator; and
(c) adding a second microsphere after reconstitution, and amplifying for 20 min at 37-44° C., wherein the second microsphere comprises a magnesium salt activator.

9.-14. (canceled)

15. The microsphere preparation according to claim 1, wherein each gram of the reaction microspheres comprises: 460.53-464.19 μg of DNA polymerase, 0.013 nmol of each primer, 657.89-663.13 μg of creatine kinase, 0.0026-0.015 mmol of DTT, 0.1-0.5 mmol of phosphokinase, 10.53-10.61 mg of single-strand binding protein, 1.71-1.72 mg of recombinase, 1.05-1.06 mg of auxiliary protein, 394.74-397.88 mg of maltose, 150-151.19 mg of PEG, 0.15 μmol of ATP, 0.01 μmol of each dNTP and 0.5 μmol of Tris-Ac.

16. The microsphere preparation according to claim 1, wherein the microsphere preparation comprises 657.89-663.13 μg of reverse transcriptase.

17. The preparation method according to claim 7, wherein the pre-freezing step is performed at a temperature of less than or equal to −54° C., with a treatment period of 0.5-1 h.

18. The preparation method according to claim 7, the main drying step is performed at a temperature of −27 to ˜15° C., with a treatment period of 2-6 h and a vacuum degree of 0.01-30 Pa; and
the final drying step is performed at a temperature of 0-20° C., with a treatment period of more than or equal to 2 h and a vacuum degree of 0.01-1 Pa.

19. A method for nucleic acid amplification coupled with a second reaction, comprising using a microsphere preparation:
wherein the microsphere preparation comprises the microsphere preparation according to claim 1;
a method of nucleic acid amplification comprises the following steps: adding the reaction microspheres into an amplification sample solution according to an addition ratio of 0.263-6.58 mL of the amplification sample solution to each gram of the reaction microspheres, and then performing amplification according to any one of the following procedures (a)-(c):
(a) adding an aqueous reconstitution solvent and an activator, mixing uniformly, and directly amplifying for 20 min at 37-44° C.;
(b) after adding a second microsphere, amplifying for 20 min at 37-44° C., wherein the second microsphere comprises a reconstitution solvent PEG and a magnesium salt activator; and
(c) adding a second microsphere after reconstitution, and amplifying for 20 min at 37-44° C., wherein the second microsphere comprises a magnesium salt activator;
all preparations used in the second reaction are microsphere preparations;
the second reaction comprises a fluorescence reaction or a CRISPR reaction.

20. The method according to claim 19, wherein the fluorescence reaction comprises adding an exonuclease and a probe into an RPA or RAA system prior to lyophilizing, so that the RPA or RAA fluorescence reaction can be detected in real time:
the exonuclease is selected from exonuclease III;
the CRISPR reaction comprises a Cas12 CRISPR detection system and a Cas 13 CRISPR detection system.

21. The method according to claim 19, wherein a method of the second reaction comprises adding the microsphere preparation for the second reaction directly into the amplification product to complete the second reaction.

22. The method according to claim 19, wherein the second reaction is a CRISPR reaction, and a preparation method for the microsphere preparation used in the CRISPR reaction comprises: preparing a CRISPR lyophilizing system, and then preparing the microsphere preparation used in the CRISPR reaction:
the CRISPR lyophilizing system comprises: 10 μL/test of CRISPR lyoprotectant, 1× Buffer, 40-100 nmol/L of Cas 12 protein, 40-100 nmol/L of Cas13 protein, 5 U of mRNase inhibitor, 14 U of T7 RNA polymerase, 0.5-0.6 mM of rNTP, 0.1 μM of CrRNA1, 0.8-1.2 nmol/L of ssDNA and 0.8-1.2 nmol/L of ssRNA.

23. The method according to claim 22, wherein the Cas 12 protein is selected from at least one of LbCas 12a, FnCas12a, AsCas12a (cpf1), BbCas12a (cpf1), and HkCas12a (cpf1):
the Cas13 protein comprises LwaCas13a;
the T7 RNA polymerase is derived from Escherichia coli.

24. A method of a fluorescent amplification reaction, comprising using the microsphere preparation according to claim 1, wherein each gram of the microsphere preparation further comprises 0.4613-12.13 mg of an exonuclease.

25. The method according to claim 24, wherein each gram of the microsphere preparation comprises 0.6579-0.6667 mg of the exonuclease.

26. A method for detection of a virus infection, comprising administering an effective amount of the microsphere preparation according to claim 1 to subject in need thereof.

27. The method according to claim 26, wherein the virus comprises at least one of the following: respiratory syncytial virus, influenza A virus, or influenza B virus.


SEQUENCE LISTING (13 sequences)

SEQ ID NO: 1 - DNA, 50 bp, synthetic construct
  source 1..50: mol_type = other DNA
  misc_binding join(35,38): bound_moiety = 35-FAM,38-BHQ1
  modified_base 36: mod_base = OTHER; note = tetrahydrofuran
ccgttggatggtgtatttgctggatgacagaagttnatctttgttgagtg

SEQ ID NO: 2 - DNA, 33 bp, synthetic construct
  source 1..33: mol_type = other DNA
atggctcttagcaaagtcaagttgaatgataca

SEQ ID NO: 3 - DNA, 32 bp, synthetic construct
  source 1..32: mol_type = other DNA
gtttctgcacatcataattaggagtatcaata

SEQ ID NO: 4 - DNA, 45 bp, synthetic construct
  source 1..45: mol_type = other DNA
  modified_base 28: mod_base = OTHER; note = tetrahydrofuran
  misc_binding join(27,29)bound_moiety
tctcctgtactacgttgaatagtgtatntgctggatgacagcagc

SEQ ID NO: 5 - DNA, 26 bp, synthetic construct
  source 1..26: mol_type = other DNA
gcacatcataattaggagtatcaata

SEQ ID NO: 6 - DNA, 54 bp, synthetic construct
  source 1..54: mol_type = other DNA
taatacgactcactatagggatgagycttctaacygargtcgaaacgtacgttc

SEQ ID NO: 7 - DNA, 30 bp, synthetic construct
  source 1..30: mol_type = other DNA
cgtctacgctgcagtccycgctcactgggc

SEQ ID NO: 8 - DNA, 35 bp, synthetic construct
  source 1..35: mol_type = other DNA
ttacactgttggttyggtgggaaagaatttgacct

SEQ ID NO: 9 - DNA, 32 bp, synthetic construct
  source 1..32: mol_type = other DNA
ttcttttttgttgctgttgtycccattccyga

SEQ ID NO: 10 - RNA, 64 nt, synthetic construct
  source 1..64: mol_type = other RNA
gatttagactaccccaaaaacgaaggggactaaaacgacaagaccaatcctgtcacctctgact

SEQ ID NO: 11 - RNA, 44 nt, synthetic construct
  source 1..44: mol_type = other RNA
taatttctactaagtgtagatgtatatcagttaagcatcttttg

SEQ ID NO: 12-13 - skipped (2 entries)